(19) United States
(12) Patent Application Publication
(10) Pub. No.: US 2025/0376672 A1
(43) Pub. Date: Dec. 11, 2025

(54) LASER ACTIVATION CHAMBER FOR CELL PRODUCTS AND BODY FLUIDS

(71) Applicant: Fariborz Izadyar, Irvine, CA (US)

(72) Inventor: Fariborz Izadyar, Irvine, CA (US)

(21) Appl. No.: 18/970,025

(22) Filed: Dec. 5, 2024

Related U.S. Application Data
(60) Provisional application No. 63/657,588, filed on Jun. 7, 2024.

(51) Int. Cl. C12N 13/00; G16H 20/10

(52) U.S. Cl. CPC C12N 13/00; G16H 20/10

(57) ABSTRACT

The present invention provides an automated and programable device equipped with a plurality of laser diodes. The device includes a housing which contains a closed chamber configured to receive and hold at least one container enclosure, such as a tube, a vial, a syringe, or a catheter, containing a sample including cells or body fluids. The device may include at least one control means configured to provide multiple settings for the plurality of laser diodes for activation of platelets, blood, plasma, and stem cells in a sample. The plurality of laser diodes may include four low level laser diodes (350, 650, 810 and 900 nm). The device may provide four pre-programmed settings for activation of platelets, blood, plasma, and stem cells in a sample. The device further has the capability to set up and save specific protocols for predetermined purposes, such as, for example without limitation, therapeutic and investigational purposes.

### RELATED APPLICATION

This application claims priority to U.S. Provisional Patent Application 63/657,588 titled “LASER ACTIVATION CHAMBER FOR CELL PRODUCTS AND BODY FLUIDS,” filed Jun. 7, 2024, the entire contents of which is incorporated herein by reference.

### BACKGROUND OF THE INVENTION

The embodiments described herein relate generally to medical devices, and more particularly to a laser activation chamber device for cell products and body fluids, and a system providing a downloadable or web-based computer application which may be used to control a laser activation chamber device.

The subject matter of this application is related to U.S. Pat. No. 11,965,179 titled “Laser Treated Platelet Product”, which claimed priority to U.S. Provisional Patent Application 63/041,863 filed Jun. 20, 2020, the entire contents of both being incorporated herein by reference. The invention described in U.S. Pat. No. 11,965,179 relates generally to medical treatments and provides a method of creating a pooled platelet lysate product from whole blood, and more particularly to a slow-release, cell-free, highly pure, laser-treated, platelet-based product for orthopedic applications for patient medical treatment.

A problem remains that existing devices and methods available are mainly made for research applications and for treatment of the body parts of patients. No tools currently exist for clinicians to address both therapeutic and investigational purposes, particularly tools which may be used for activation of cells or body fluids for therapeutic or investigational purposes.

Further, there is currently no existing automated low level laser activation chamber for cell products and body fluids, wherein such chamber may be used by clinicians or investigators to develop new laser activation modules for specific cell types or body fluids to achieve a specific goal. Currently, no tools exist for clinicians to stimulate the cell products of their patients, either in a syringe format or in a tube.

In addition, no laser activation chamber exists which enables a user to control and adjust laser intensity, time of laser exposure, and the mode of laser. No device exists which is capable of radiating low level laser with three wavelengths as single exposure or in combination. No system exists providing a downloadable or web-based computer application which may be used to control a laser activation chamber device.

Therefore, what is needed is a solution to one or more of these problems.

### SUMMARY OF THE INVENTION

Some embodiments of the present disclosure include a laser activation chamber device for cell products and body fluids. In one implementation, the present invention may provide an automated and programmable device comprising an automated low level laser activation chamber for cells, cell products, or body fluids. The device may comprise a housing which contains a closed chamber configured to receive and hold container enclosures such as tubes, vials, and syringes, which contain samples comprising cells or body fluids. In one aspect, the container enclosures are translucent, allowing laser light to effectively reach the cells or fluids therein.

Cells, cell products and body fluids which may be activated by the device include mammalian cells such as human cells, dog cells, cat cells or horse cells. The cells may comprise blood cells including platelets or white blood cells (WBCs); somatic cells including skin cells, muscle cells, or stem cells; or stem cells including hematopoietic stem cells, mesenchymal stem cells, pluripotent stem cells, or germline stem cells. The body fluid may comprise serum, plasma, follicular fluid, seminal plasma, or other body fluids that hold or support cells within them, and combinations thereof.

The laser activation chamber is equipped with a plurality of laser diodes and at least one control means configured to provide multiple settings for activation of a sample. The device may provide pre-programmed settings for activation of platelets, blood, plasma, and stem cells in the sample. The device further has the capability to set up and save specific protocols for predetermined purposes, such as, for example without limitation, therapeutic purposes, and investigational purposes. The device has the ability to provide single-laser or multiple-laser exposure of a sample, a predetermined time of exposure, and a predetermined mode of exposure. The device may supply a plurality of laser beams, wherein each laser beam of said plurality of laser beams may be adjusted independently. Further, the time of exposure and the mode of exposure for each laser beam of said plurality of laser beams may be adjusted independently. The device allows the investigators to develop new laser activation modules for specific cell types or body fluids to achieve a specific goal.

The device is configured to use electric power (DC) and may also function as a portable device using AC rechargeable batteries.

In another implementation, the present invention provides a laser activation chamber system comprising an automated low level laser activation chamber device as described herein, and a downloadable or web-based computer application which may be used to control the device. The computer application may be configured to communicate with a computer control module of the device, and a device control panel. The computer application may be a custom application provided to a user in the form of software as a service (SAAS).

### BRIEF DESCRIPTION OF THE FIGURES

The detailed description of some embodiments of the invention is made with reference to the accompanying figures, wherein like numerals represent corresponding parts of the figures.

FIG. 1A conceptually illustrates a front right perspective view of a laser activation chamber device in accordance with an embodiment of the present invention;

FIG. 1B conceptually illustrates a top view of the laser activation chamber device as in FIG. 1A;

FIG. 1C conceptually illustrates a back view of the laser activation chamber device as in FIG. 1A;

FIG. 1D conceptually illustrates a front view of the laser activation chamber device as in FIG. 1A;

FIG. 2 conceptually illustrates a block diagram of the laser activation chamber device;

FIG. 3 conceptually illustrates a schematic diagram of the components of the laser activation chamber system for cell products and body fluids in accordance with an embodiment of the present invention;

FIG. 4A conceptually illustrates a schematic front isometric view of a laser chamber of the system as in FIG. 3;

FIG. 4B conceptually illustrates a schematic top view of the laser chamber as in FIG. 4A;

FIG. 5 conceptually illustrates a flow chart of the measurement of laser and temperature by the laser activation chamber system;

FIG. 6 conceptually illustrates a flow chart of the measurement of the platelet morphology/aggregation by the system; and

FIG. 7 conceptually illustrates a flow chart of the measurement of the start/finish/stop/process of the system.

### DETAILED DESCRIPTION OF CERTAIN EMBODIMENTS

In the following detailed description of the invention, numerous details, examples, and embodiments of the invention are described. However, it will be clear and apparent to one skilled in the art that the invention is not limited to the embodiments set forth and that the invention can be adapted for any of several applications.

Some embodiments of the present disclosure include a laser activation chamber device and system for cells, cell products and body fluids. The device and system may be used as an automated low level laser activation chamber device and system, and may comprise the following elements related in the following exemplary fashion. This list of possible constituent elements and their interrelation described herein are intended to be exemplary only, and are not intended to be used to limit the device of the present application to just these elements or to limit the scope or nature of the relationships between the various elements Persons having ordinary skill in the art relevant to the present disclosure may understand there to be equivalent elements that may be substituted within the present disclosure without changing the essential function or operation of the device. The following examples are presented as illustrative examples only.

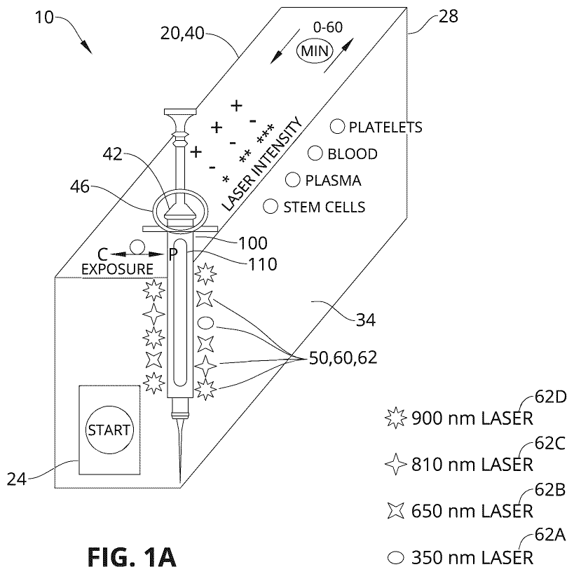

Referring initially to FIGS. 1A-D and 2, the present invention provides an automated low level laser activation chamber device 10 for laser activation of cells, cell products and body fluids (hereinafter the device 10). The device 10 may comprise a housing 20 having a front 24, a back 28, a top 30, a bottom 32, a first right side 34 and a second left side 36. The housing 20 may define a chamber 40 having an interior 42, the chamber 40 having at least one slot or well 46 having a circular opening, the at least one slot or well 46 being configured to removably receive and retain at least one container enclosure 100 containing a sample 110 comprising somatic cells, cell products, or body fluids.

A sample may comprise somatic cells or body fluids from mammals. The sample may comprise mammalian cells, such as, from a human, a dog, a cat, or a horse. The sample may comprise blood cells, skin or muscle cells, or stem cells. The blood cells may comprise platelets or white blood cells (WBCs). The stem cells may comprise hematopoietic stem cells, mesenchymal stem cells, pluripotent stem cells, and combinations thereof. The sample may comprise serum, plasma, follicular fluid, seminal plasma, or other body fluids that hold or support cells within them.

The at least one container enclosure 100 may be selected from a tube, a vial, a syringe, or combinations thereof. The container enclosures may be customized to contain either cells or body fluids. In some embodiments, the at least one slot or well 46 may be configured to hold a 3-10 ml syringe or a 3-30 ml conical tube. The at least one slot or well 46 may further be configured to accommodate a catheter passing through the chamber, or at least a portion of a tube passing through the chamber, for continuous flow of fluids during activation. In some embodiments, the chamber 40 is configured to receive one container enclosure 100 containing a sample 110.

The device 10 may be equipped with at least one laser light source 60 located in the housing 20, the laser light source 60 being configured for activation of at least one of platelets, blood, plasma, stem cells, or combinations thereof, in the sample 110 contained in the at least one container enclosure 100. The laser light source 60 may comprise a plurality of laser diodes 62 configured to emit laser light having a predetermined wavelength of between about 350 and 900 nm. The plurality of laser diodes 62 may comprise a first laser diode 62A configured to emit laser light having a wavelength of 350 nm, a second laser diode 62B configured to emit laser light having a wavelength of 650 nm, a third laser diode configured to emit laser light having a wavelength of 810 nm and a fourth laser diode configured to emit laser light having a wavelength of 900 nm. In some embodiments, the at least one slot or well 46 is surrounded by laser beams emitted from the plurality of laser diodes 62 of different wavelengths. The position of the laser diodes 62 may be adjustable with respect to the sample 110 to provide a distance between 2 and 4 inches.

The device 10 may provide pre-programmed settings for activation of platelets, blood, plasma, and stem cells in a sample. The device 10 may further have the capability to set up and save specific protocols for activation of cells or body fluids for predetermined purposes, such as, for example without limitation, therapeutic purposes and investigational purposes. The device 10 may have the ability to provide single-laser or multiple-laser exposure of the sample 110, a predetermined time of exposure between 0 and 60 minutes, and a predetermined mode of exposure selected from continuous mode or pulsed mode. The laser diodes 62 may be adjusted independently to change the mode, intensity, and duration of laser exposure.

The device 10 may permit a clinician or an investigator to develop and program new laser activation modules for specific cell types or body fluids to achieve a specific goal. Laser intensity, mode of exposure, and duration may have a significant effect on the final outcome of laser activation. For instance, the rate of proliferation or differentiation of stem cells may be affected by a specific laser activation protocol. Laser activation may be used to awaken quiescent stem cells, revive senescent stem cells, and/or modify components of the body fluid, by activating certain molecules, peptides, or receptor proteins circulating in the blood or other body fluids.

The device 10 may comprise at least one control means 70 configured to provide a plurality of settings for the plurality of laser diodes 62 for activation of the sample in the at least one container enclosure 100. The at least one control means 70 may be a pre-programmed settings control 76 configured to provide four pre-programmed settings capable of selection for activation of somatic cells, platelets, blood cells, plasma, and stem cells in a sample 110. The at least one control means 70 may include at least one laser diode selection control 80 configured to enable a user to select at least one laser diode 62 or a plurality of laser diodes 62 to provide a single-laser exposure or a multiple-laser exposure of a sample 110. The at least one laser diode selection control 80 may be configured to enable a user to independently control the first laser diode 62A, the second laser diode 62B, the third laser diode 62C, and the fourth laser diode 62D to select and adjust a time of operation from zero to about sixty (60) minutes, and/or to select a mode of operation between a continuous mode and a pulsed mode. The at least one control means 70 may comprise a timer control that may be used to adjust the duration of exposure for each laser diode 62 independently. The at least one control means 70 may provide a laser exposure mode that may be adjusted for each laser and a laser intensity control which may also be adjusted for each laser diode 62 and may further be configured to enable the user to program and save at least one specific protocol for activation of a sample for a predetermined purpose selected from therapeutic purposes, investigational purposes, and combinations thereof. The at least one control means 70 may comprise a laser meter, a thermometer, a camera, and a light scatter spectrophotometer. As described herein, the camera may comprise a high resolution, high powered wireless lens sensitive to and capable of detecting features of a sample such as, for example without limitation, cellular morphology, platelet morphology, platelet aggregation, or the like.

As shown at TABLE 1, features and functions of the plurality of lasers of the device 10 are shown and described, and indicate various protocols and parameters for continuous exposure, pulsed exposure, and combined continuous and pulsed exposure of particular laser light wavelengths.

TABLE 1
LASER COMBINATION CAPABILITY

| Laser | | | |
|---|---|---|---|
| Wavelength (nm) | Continuous Exposure | Pulsed Exposure | Combined |
| 650 | 650-C | 650-P | 650-C and 650-P |
| 810 | 810-C | 810-P | 810-C and 810-P |
| 900 | 900-C | 900-P | 900-C and 900-P |
| 650 and 810 | 650-C and 810-C | 650-P and 810-P | 650-C and 810-P |
| | | | 650-P and 810-C |
| 650 and 900 | 650-C and 900-C | 650-P and 900-P | 650-C and 900-P |
| | | | 650-P and 900-C |
| 810 and 900 | 810-C and 900-C | 810-P and 900-P | 810-C and 900-P |
| | | | 810-P and 900-C |
| 650 and 810 and | 650-C and 810-C | 650-P and 810-P | 650-C and 810-C |
| 900 | and 900-C | and 900-P | and 900-P |
| | | | 650-C and 810-P |
| | | | and 900-P |
| | | | 650-P and 810-C |
| | | | and 900-C |
| | | | 650-P and 810-P |
| | | | and 900-C |

The at least one control means 70 may further include a power or on/off switch or button 86 and a start button 88. A power source 90 is operably connected to the plurality of laser diodes 62 and the at least one control means 70. The power source 90 may comprise an AC power source, a disposable DC battery power source, or a rechargeable DC battery power source that may be recharged by the AC power source. A recharging and power cord may be operably connected to the AC power source and/or the rechargeable DC battery power source. In embodiments having a recharging and power cord, the cord may be any suitable cord which includes a USB adaptor configured to operably connect to the device 10 by a USB port 22 (best seen at FIG. 1C) on the device 10 and to a conventional AC outlet (which may also have a USB receptacle). The USB port 22 may be located on the device housing 20 and may be operably connected to the battery so the USB cord may be used to recharge the battery and/or supply power directly to the device 10. The internal rechargeable battery may hold and supply power for about 8 to 24 hours. The device 10 may thus be portable.

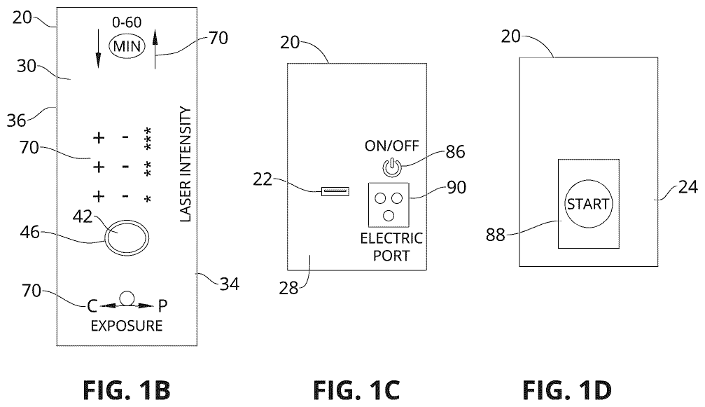

As shown at FIG. 2, the power source 90 may be operably connected to a printed circuit board (PCB) 98 and to the laser diodes 62; the laser selection controls 80; the on/off switch 86; the start button 88; and multiple controls 70, including the pre-set protocols controls and pre-set protocols database, the user-set protocols controls, a user-set protocols database, and other controls including a device timer for time of laser exposure, a temperature setting control, and a laser position control; a device alarm 92; a device automatic stop 94; and a device speaker 96. The laser selection controls 80 may also include the separate controls for laser intensity and laser exposure mode.

An example of a protocol for activation of platelet rich plasma (PRP) using the device 10 may include the following steps: turning on the device 10; adjusting the time for 15 min; adjusting the laser intensity to maximum (650+810+900); adjusting the exposure mode to continuous; inserting a sample in a container enclosure 100 into the device 10; clicking start; and after 15 min, the device 10 automatically stops and beeps, letting the user know that activation is finished.

In some embodiments, the device 10 may be used to activate platelets in PRP prior to autologous administration to a patient's own body. For this application, a tube of blood is taken from the patient, PRP is prepared using an existing PRP kit, and PRP is collected in a syringe and subjected to the device 10 for laser activation. Laser activated PRP is then injected into the patient. In another exemplary embodiment, platelets may be activated prior to preparation of platelet lysate (PL). This may be autologous, where PL is injected into a patient's own body. In this case, after platelets are concentrated, they may be activated in the chamber device 10 prior to lysate preparation. For allogeneic use, PL is made by pooling platelets from several blood donors. In this application laser activation can be done a) prior to concentrating the platelets; b) after concentrating platelets and prior to pooling; or c) after concentrating platelets and after pooling. In other embodiments, platelets could be mixed with a gel, such as hydrogel, prior to laser activation. The syringe containing gel and platelets is placed in the activation chamber, and after laser activation, gel can be applied for therapeutic use in wound healing or for local injections in muscle, intra articular administration, or other body parts.

Other protocols may be followed with the device 10. For example, WBCs may be concentrated as leuko pack and placed in the activation chamber. After activation, the WBCs can be used for therapeutic use. In another embodiment, somatic cells such as skin cells or muscle cells may be placed in the chamber for laser activation prior to therapeutic use. In yet further embodiments, stem cells such as hematopoietic stem cells, mesenchymal stem cells, germline stem cells. or pluripotent stem cells may be placed in the laser activation chamber 40 to boost their therapeutic potency or differentiation capacity. In further embodiments, body fluids may be exposed to the laser activation chamber 40, by placing a small sample in the insert or by continuously passing a sample through a tube, at least a portion of which is positioned in the activation chamber 40. In some embodiments, the device 10 may be used as a dialysis machine, to expose blood to at least one laser beam using the device 10 while the blood is outside of a patient's body. Implementing the device 10 in connection with dialysis may potentially boost the energy level of a patient's blood cells and increase the immunity level of the patient. The device 10 may be used as an accessory tool to a current dialysis machine in a protocol exposing the fluid part of a patient's blood to the device 10 to reduce toxic compounds such as bilirubin, and to enhance ATP to improve patient's condition, which may reduce the time and frequency of dialysis in some patients. The laser activation device chamber may be operably connected to an existing dialysis machine.

In some embodiments, the device 10 may be used for research to study the effect of individual or a combination of lasers on cells including plant cells and microorganisms, and on other molecules that are present in a liquid form. In other embodiments, the device 10 may be used as a laser activation chamber 40 for injectable cells. The device 10 may also be used in connection with the treatment methods of creating a pooled platelet lysate product of U.S. Pat. No. 11,965,179.

The device 10 and its components may be made of any suitable materials and may be fabricated by any suitable fabrication process. Nonlimiting examples of materials of which the housing 20 and the chamber 40 may be made include metals, hard plastics, and combinations thereof. The housing 20 and the chamber 40 may be made of a material that is capable of keeping the chamber interior 42 dark to protect the sample therein from light and prevent laser light exposure outside of the chamber 40, as a safety feature. The container enclosures 100 may be made of a translucent or clear material capable of permitting laser light to effectively reach the sample 110 therein.

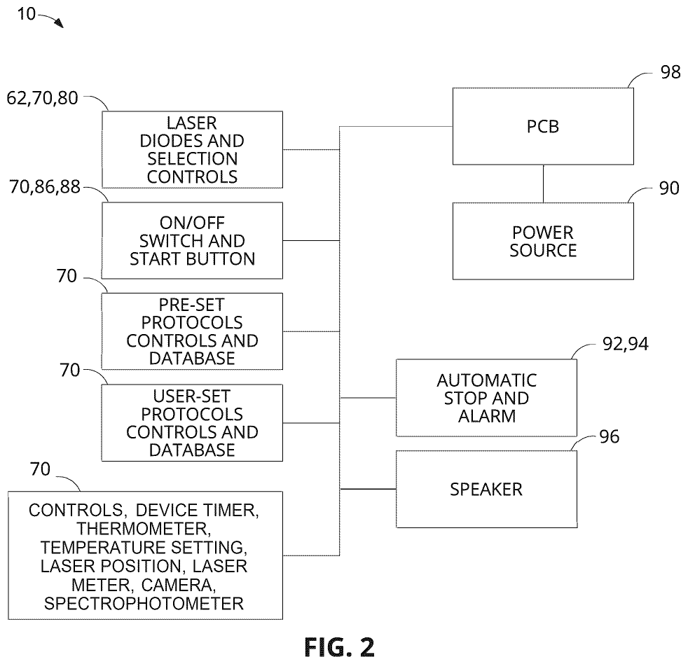

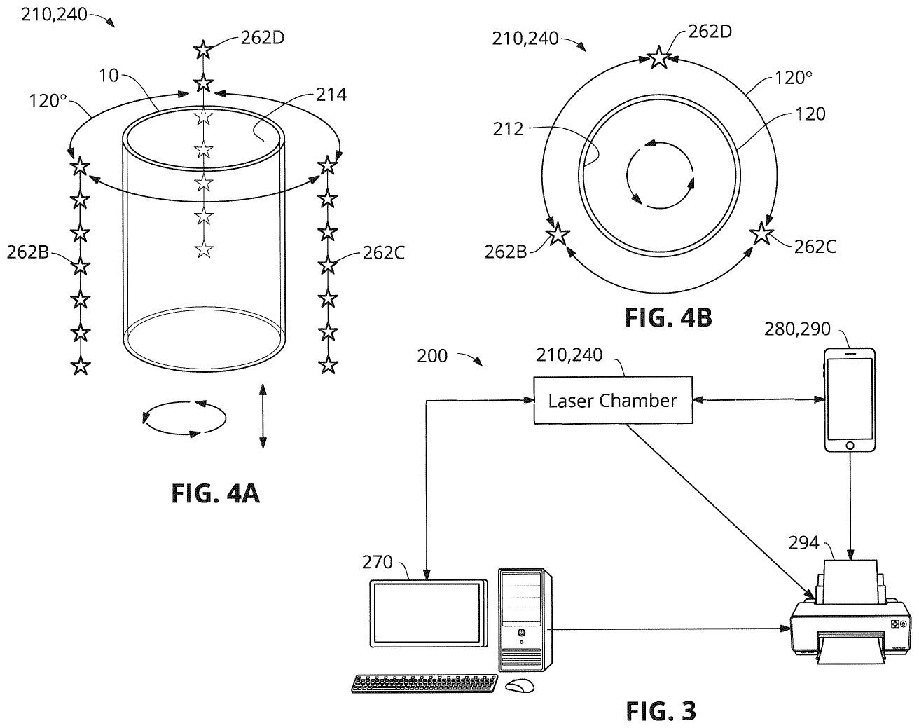

Referring now to FIGS. 3 and 4A-B, in another implementation, the present invention may provide a system 200 for laser activation of cells, cell products and body fluids. Reference numerals for elements of the device 10 heretofore described in the 10-199 series with respect to FIGS. 1A-D and 2, are designated by the same reference numerals in 200-399 series of FIGS. 3 and 4A-B schematically showing the system 200. The system 200 may include an automated low level laser activation chamber device 210. In some embodiments the system 200 may comprise a device 10 as described hereinabove with reference to FIGS. 1A-D and 2.

The device 210 may include a laser activation chamber 240 with a sample insert 212 for holding a sample, the sample insert 212 having an interior reflective surface 214. The device 210 may include a plurality of lasers 262 including a 650-nanometer laser 262B, an 810-nanometer laser 262C, and a 900-nanometer laser 262D. The system 200 further comprises a computer control module 270 and a system computer software program/application 280 (hereinafter system software application 280). The system software application 280 may be a downloadable or web-based application configured for use with the computer control module (CCM) 270 and a device control panel which may include controls as described with reference to FIG. 2 hereinabove. The system software application 280 may be installed on a mobile device 290 and may further comprise a printer 294. In some embodiments, the system software application 280 may be a custom application provided in the form of software as a service (SAAS) configured to provide specific functions of the system 200 and the device 210 as described herein and in accordance with the user's needs. The customer may purchase a SAAS system configured to provide automatic access to at least one selected function, and may be provided automatic access to the selected function. The system may provide control of the position or specifically positioned lasers 262 or sample insert 212, a laser meter, a thermometer, platelet morphology, the computer control module and artificial intelligence (AI).

The position of the laser beams and the sample insert are shown at FIGS. 4A-B. The laser diodes 262 may be constructed and arranged to emit laser beams that are positioned in three lines around the sample insert 212, at 1-2 inches from the sample insert 212, such that the laser beams emitted from the laser diodes 262B,262C,262D are positioned 120 degrees from each other. The sample insert 212 is constructed and arranged for rotational movement and vertical movement up and down, to expose all the cells, cell products or body fluids of a sample present in a container enclosure with equal amount, intensity, and duration of laser.

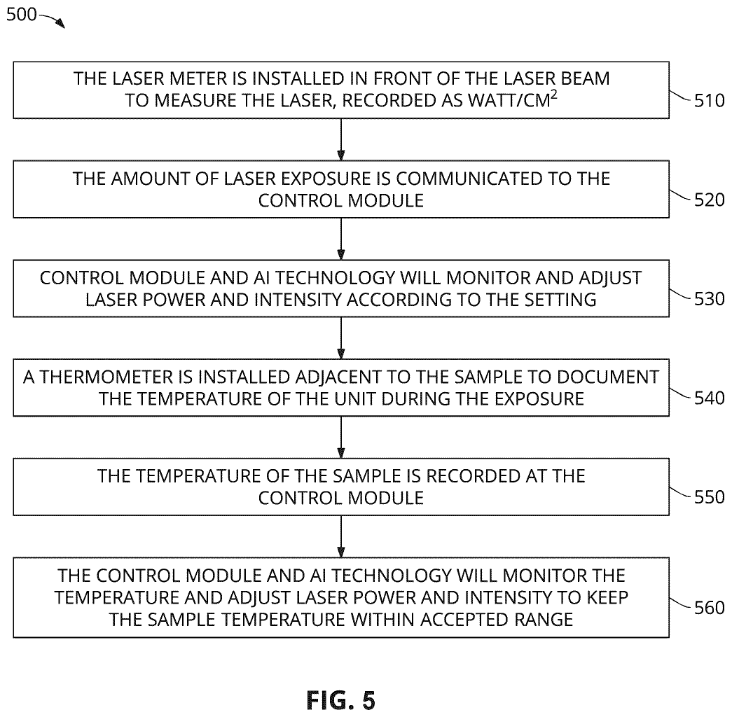

Referring to FIG. 5, a flow chart shows a method 500 for the measurement of laser and temperature by the device 210 and the system 200. The method 500 may include the following steps. 510: installing the laser meter in front of the laser beam trajectory to measure the laser exposure to a sample, which is recorded in watts per square centimeter (W/cm²). 520: communicating the amount of laser exposure to the CCM 270. 530: monitoring and adjusting the laser power and intensity according to the system setting using the CCM and AI technology. 540: installing a thermometer adjacent to the sample to measure and document the temperature of the sample insert 212 during the exposure. 550: recording the temperature of the sample at the control module. 560: monitoring the temperature and adjusting the laser power and intensity using the CCM 270 and AI technology to keep the sample temperature within an accepted range between 25 and 38° C.

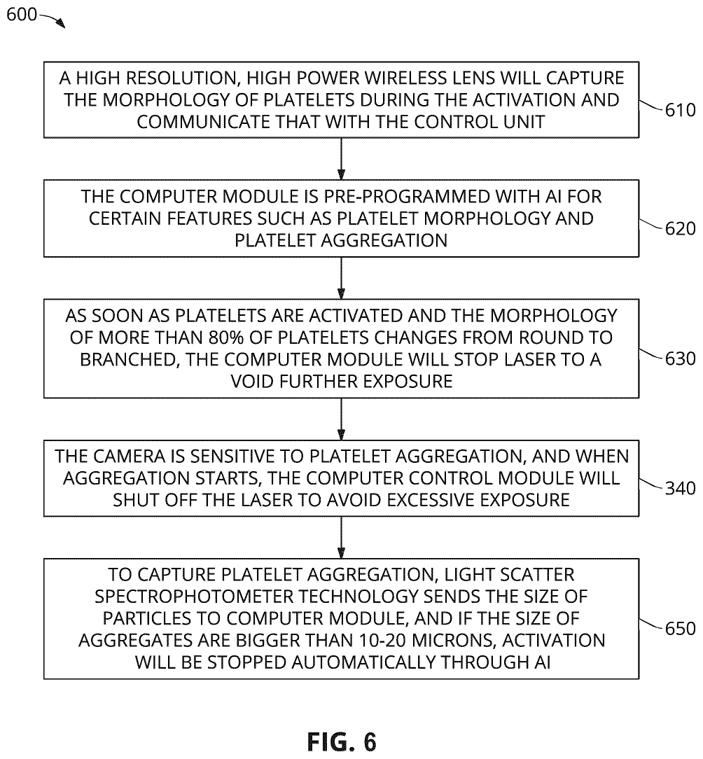

FIG. 6 presents a flow chart of a method 600 for the measurement of the platelet morphology/aggregation by the device 110 and the system 200. The method 600 may include the following steps. 610: capturing the morphology of platelets during the activation using a high resolution, high power wireless lens, and communicating that data with the control unit of the system. 620: pre-programming the CCM 270 with AI for certain features such as platelet morphology and platelet aggregation. 630: stopping the laser using the CCM 270 as soon as platelets are activated and the morphology of more than 80% of the platelets changes from round to branched to avoid further exposure to the platelets. 640: in response to detection by the camera (having sensitivity to platelet aggregation) of aggregation starting, the computer control module will shut off the laser to avoid excessive exposure. 650: performing light scatter spectrophotometer technology to capture platelet aggregation, sending the size of particles data to the CCM 270, and stopping activation automatically through AI if the size of aggregates is larger than 10-20 microns.

The CCM 270 may be programmed through a touch screen at the side of the laser activation chamber device 210 or through a system software application 280 which may be downloaded to a computer or a mobile device 290. The system software application 280 is configured for wireless communication with the CCM 270 and the printer 294. The system software application 280 is configured to provide a clinical mode or an investigational mode and may provide modules for each specific clinical use, such as platelet applications, stem cell applications, body fluid applications, or the like. The clinical mode provides preprogrammed settings for platelets, stem cells, blood, or body fluid. The investigational mode is configured to provide investigation of cell therapy tools and for developing new programs. For the investigational mode, the settings need to be specified by the user. The system software application 280 is configured to document the laser treatment in several pages: page 1—clinical mode or investigational mode; page 2—patient/sample information and laser configuration; page 3—start/finish; page 4—report (print/save). The user will choose the sample type, insert the sample, and operate the device 210 using the system 200. The user will select a clinical mode preprogrammed setting or select investigational mode and specify the settings. The system 200 will show the temperature (° C.) and the laser exposure (W/cm²) at an upper portion of the screen. The system 200 will enable the user to enter the name of a patient/sample, the sample type (blood, PRP, stem cells, body fluid, or the like), and the date/time of the treatment.

The laser settings will be selected, with exemplary laser settings including the following:

| | | | |
|---|---|---|---|
| 650 nm | Continuous/pulsed | Duration (min) | Intensity (L/M/H) |
| 810 nm | Continuous/pulsed | Duration (min) | Intensity (L/M/H) |
| 900 nm | Continuous/pulsed | Duration (min) | Intensity (L/M/H) |

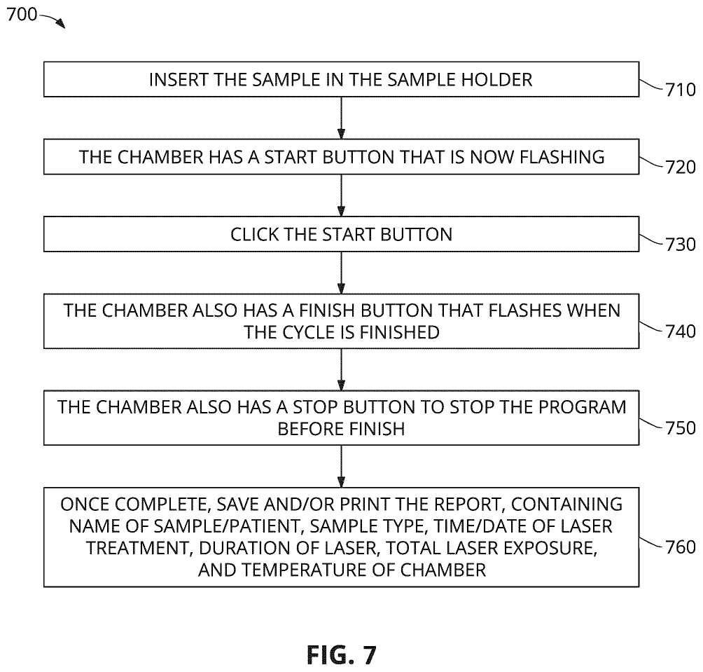

Referring now to FIG. 7, a flow chart shows a method 700 for the start/finish stop process and the save/print data and measurements of the system 200 and device 210. The method 700 may include the following steps: 710: inserting a sample in the sample insert 212, causing 720: the start button on the chamber to start flashing. 730: actuating the start button to start the device. 740: allowing the cycle to finish, causing a finish button on the chamber to flash, or 750: actuating a stop/finish button on the chamber to stop the program before the cycle is completed. 760: saving and/or printing a report once the process is complete, wherein the system includes print and save buttons. The report includes the following data: name of sample/patient; sample type; time/date of laser treatment; duration of laser (min); total laser exposure (W); and temperature of chamber: (° C.). The data may be saved on a USB that is provided along with the device and includes all the instructions.

The advantages of the device 10 and the system 200 of the present invention are many. The device 10 and system 200 provide tools to clinicians and investigators to stimulate cellular products of individuals, in a syringe, a test tube, a tube, or a catheter. The container enclosure 100 of the device 10 maintains the integrity of the cells, body fluids and plasma of the sample materials, which is ideal for therapeutic uses, and provides superior function for research and investigative applications. The device 10 and system 200 may be fully automated and programmable, and may be programmed for a particular use. The device 10 and system 200 provide a broad spectrum of lasers capable of use to radiate with at least one wavelength, with multiple wavelengths such as three to four wavelengths during a single exposure, or during multiple exposures in a single process. The controls 70 provide the ability to select and customize various parameters independently for each laser diode and laser beam. The device 10,210 may be configured to operate at cold (4° C.) and room (20-25° C.) temperatures. The device 10 and system 200 may be operable with 120V or 220V DC power and rechargeable batteries. These features of the device 10 and system 200 differ from and distinguish over previous solutions and provide improved functionality.

The functions described above can be implemented in digital electronic circuitry, computer software, firmware, or hardware. The techniques can be implemented using one or more computer program products. Programmable processors and computers can be packaged or included in mobile devices. The processes may be performed by one or more programmable processors and by one or more set of programmable logic circuitry. General and special purpose computing and storage devices can be interconnected through communication networks. Some embodiments include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable discs, ultra-density optical discs, any other optical or magnetic media, and floppy disks. The computer-readable media may store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the invention has been described with reference to numerous specific details, persons of ordinary skill in the art may appreciate that numerous design configurations may be possible to enjoy the functional benefits of the inventive systems, and the invention can be embodied in other specific forms without departing from the spirit of the invention. Specific method steps may not be performed in the exact order shown and described. Specific operations may not be performed in one continuous series of operations, and different specific operations may be performed in different embodiments. Furthermore, the methods described herein, such as methods 500,600,700, could be implemented using several sub-processes, or as part of a larger macro process. Thus, one of ordinary skill in the art would understand that the invention is not to be limited by the foregoing illustrative details, but rather is to be defined by the appended claims.

What is claimed is:

1. An automated programmable laser activation device for laser activation of somatic cells, blood cells, platelets, plasma, stem cells, cell products, and body fluids, the device comprising:
a laser activation chamber having an interior portion configured to removably receive and retain at least one container enclosure containing a sample;
a plurality of laser diodes constructed and arranged about the laser activation chamber for laser activation of the sample;
at least one control means operatively connected to the plurality of laser diodes, the at least one control means being configured to provide a plurality of settings for operation of the laser activation chamber and the plurality of laser diodes; and
a power source operably connected to the plurality of laser diodes and the at least one control means.

2. The laser activation device of claim 1, wherein:
the laser activation chamber is made of at least one material selected from the group consisting of metals, hard plastics, and combinations thereof; and
the at least one material is capable of keeping the interior portion dark to protect the sample from light and preventing laser light exposure outside of the laser activation chamber.

3. The laser activation device of claim 2, wherein the at least one container enclosure is made of a translucent or clear material capable of permitting laser light to reach the sample therein.

4. The laser activation device of claim 1, wherein each laser diode of the plurality of laser diodes is configured to emit laser light having a wavelength between 350 and 900 nm.

5. The laser activation device of claim 1, wherein the plurality of laser diodes comprises a 650-nm laser diode, an 810-nm laser diode and a 900-nm laser diode.

6. The laser activation device of claim 5, wherein each laser diode of the plurality of laser diodes is capable of generating laser power of at least 10-100 mW/second.

7. The laser activation device of claim 1, wherein the at least one control means is configured to provide a plurality of pre-programmed settings capable of selection by a user for laser activation of somatic cells, blood cells, platelets, plasma, stem cells, cell products, or body fluids in the sample.

8. A system for laser activation of somatic cells, blood, platelets, plasma, stem cells, cell products, and body fluids, the system comprising:
a laser activation device comprising:
a laser activation chamber having an interior portion configured to removably receive and retain at least one container enclosure containing a sample;
a sample insert configured for removable placement in the interior portion of the laser activation chamber, the sample insert having an interior reflective surface;
a plurality of laser diodes constructed and arranged about the laser activation chamber for laser activation of the sample;
at least one control means operatively connected to the plurality of laser diodes, the at least one control means being configured to provide a plurality of settings for operation of the laser activation chamber and the plurality of laser diodes; and
a power source operably connected to the plurality of laser diodes and the at least one control means;
a computer control module programmed with a laser activation system artificial intelligence (AI); and
a system computer software program configured for use with the computer control module to operate the at least one control means of the laser activation device and the system computer software program.

9. The system of claim 8, further comprising a system printer operably connected to the laser activation device, the computer control module, and the system computer software program.

10. The system of claim 8, wherein the plurality of laser diodes further comprises a 650-nm laser diode, an 810-nm laser diode and a 900-nm laser diode.

11. The system of claim 10, wherein the laser activation device further comprises a laser meter and a thermometer.

12. The system of claim 11, wherein the laser activation chamber further comprises a wireless lens configured to capture image data of a sample during activation and wirelessly transmit the image data to the computer control module.

13. The system of claim 12, wherein:
the sample comprises platelets;
the wireless lens is sensitive to platelet aggregation;
the image data comprises platelet morphology image data of the sample during activation;
the AI is programmed to monitor and analyze platelet morphology and detect platelet aggregation; and
if, during laser activation of the sample, in response to detection by the AI that the morphology of more than 80% of the platelets of the sample has changed from a round morphology to a branched morphology, the computer control module will automatically stop the laser activation to avoid further laser exposure to the sample.

14. The system of claim 13, wherein:
the wireless lens is configured to perform light scatter spectrophotometry and transmit size of particles data to the computer control module;
the AI is programmed to analyze the size of particles data for a plurality of platelet aggregates detected in the sample, and determine whether any platelet aggregates present in the plurality of platelet aggregates have a dimension of greater than about 10 to 20 microns; and
the AI is programmed to automatically stop the laser activation in response to a determination that platelet aggregates having a dimension of greater than about 10 to 20 microns are present in the sample.

15. The system of claim 10, wherein:
the 650-nm laser diode, the 810-nm laser diode, and the 900-nm laser diode are positioned 120 degrees apart, are constructed and arranged to emit laser beams 120 degrees from each other, and are each positioned between 1 to 2 inches from the sample insert; and
the sample insert is constructed and arranged for rotational movement and vertical movement up and down, to expose an entirety of the sample to an equal amount, intensity, and duration of laser energy.

16. The system of claim 15, wherein the system computer software program is configured to operate the at least one control means of the laser activation device to select at least one function from the group consisting of control of the position of the plurality of laser diodes, control of the position of the sample insert, operation of the laser meter, and operation of the thermometer.

17. The system of claim 9, wherein the system computer software program is provided as software as a service (SAAS).

18. The system of claim 9, wherein the system software application is downloaded or installed on a computing device.

19. The system of claim 18, wherein the computing device comprises a mobile device.

20. The system of claim 16, wherein:
the at least one control means is configured to provide a plurality of pre-programmed settings capable of selection for activation of somatic cells, platelets, blood cells, plasma, and stem cells in a sample;
the at least one control means is configured to enable a user to select at least one laser diode or a plurality of laser diodes to provide a single-laser exposure or a multiple-laser exposure of a sample;
the at least one control means is configured to enable a user to independently control the 650-nm laser diode, the 810-nm laser diode, and the 900-nm laser diode, to select and adjust a time of operation between zero to sixty (60) minutes, and to select a mode of operation between a continuous mode and a pulsed mode; and
the at least one control means is configured to enable the user to program and save at least one specific protocol for activation of a sample for a predetermined purpose selected from the group consisting of therapeutic purposes, investigational purposes, and combinations thereof.

21. The system of claim 20, wherein the plurality of pre-programmed settings further comprises:
a first pre-programmed setting comprising using the 650-nm laser diode and the 810-nm laser diode and a 900-nm laser diode;
a second pre-programmed setting comprising using the 650-nm laser diode and the 900-nm laser diode;
a third pre-programmed setting comprises using the 810-nm laser diode and a 900-nm laser diode; and
a fourth pre-programmed setting comprising using the 650-nm laser diode, the 810-nm laser diode, and the 900-nm laser diode.

22. The system of claim 16, wherein:
the laser meter is constructed and arranged to measure a laser exposure to the sample, record a measured laser exposure in watts per square centimeter (W/cm²), and transmit the measured laser exposure to the computer control module;
the thermometer is configured to measure a temperature of the sample insert and transmit a measured temperature of the sample insert to the computer control module;
the AI is programmed to monitor and analyze the measured laser exposure and the measured temperature of the sample insert, and to operate the at least one control means of the laser activation device in accordance with a system setting to maintain a sample temperature within a range of between 25 and 38° C.

23. The system of claim 16, wherein the system software application is configured to document a laser activation treatment of a sample by recording data selected from the group consisting of:
whether the laser activation treatment of the sample has been conducted in a clinical mode or an investigational mode;
patient and/or sample identification information of the sample;
a sample type;
a date and time of the laser activation treatment;
a system setting and a laser configuration of the laser activation treatment;
a sample temperature in ° C., a duration of the laser activation treatment in minutes, a laser exposure to the sample in W/cm², and a total laser exposure of the sample in W;
start/finish data; and
whether documentation of the laser activation treatment has been printed and/or saved.

24. The system of claim 23, wherein:
operation of the system in the clinical mode comprises selection by a user of a pre-programmed setting for a sample;
operation of the system in the investigational mode comprises selection of a plurality of settings by the user.

25. The system of claim 24, wherein the user may provide a laser activation treatment cycle to a sample by:
inserting the sample in the sample insert, causing a start button of the laser activation chamber to flash in response to an insertion of the sample in the sample insert;
actuating the start button to start the device;
allowing the laser activation treatment cycle to finish, causing a finish button on the chamber to flash, or actuating a stop/finish button to stop the laser activation treatment cycle before the laser activation treatment cycle has been completed;
the system generating a report of the laser activation treatment; and
saving the report by actuating a save button.

26. The system of claim 25, wherein:
the system further comprises a system printer operably connected to the laser activation device, the computer control module, and the system computer software program; and
providing the laser activation treatment cycle to a sample further comprises printing the report by actuating a print button.
